(19) United States
(12) Patent Application Publication
(10) Pub. No.: US 2026/0298921 A1
(43) Pub. Date: Oct. 1, 2026

(54) Multi-Analyte Rapid Diagnostic Test for Sepsis Using Novel Biomarkers and Quantum Dot Technology

(71) Applicant: Trimar Domestic Inc., Denison, TX (US)

(72) Inventors: Douglas Fraser, Denison, TX (US); Steven Squires, Denison, TX (US); Krishna Kowlgi, Denison, TX (US); Andrew Robinson, Denison, TX (US); Brent Ferguson, Denison, TX (US)

(21) Appl. No.: 18/880,490

(22) PCT Filed: Jun. 30, 2023
(86) PCT No.: PCT/US2023/026707

§ 371 Date: Dec. 31, 2024

Related U.S. Application Data
(60) Provisional application No. 63/357,887, filed on Jul. 1, 2022.

(51) Int. Cl. G01N 33/543

(52) U.S. Cl. CPC G01N 33/54388; G01N 2333/523; G01N 2333/535; G01N 2333/5412; G01N 2333/5428; G01N 2333/585; G01N 2333/7155; G01N 2333/966; G01N 2800/26

(57) ABSTRACT

The invention encompasses rapid diagnostic testing devices for testing of a biological sample for sepsis, and for determining bacterial versus viral pathogen. The device comprises a channeled construct, at least one lateral flow unit, and a cassette housing for the detection of biomarkers indicative of sepsis. The lateral flow unit is at least partially disposed in the cassette housing. The lateral flow unit comprises: a sample receiving zone, a conjugate zone and a detection zone. The sample receiving zone is operatively coupled to the channeled construct for receiving the biological sample comprising at least one analyte (e.g., a biomarker). The conjugate zone comprising a conjugate particle to bind the analyte is disposed adjacent to the first side of the sample receiving zone. The detection zone is disposed adjacent to the second side of the sample receiving zone and comprises at least one binding agent for detecting the analyte and includes quantum dots to amplify the signal for high sensitivity.

### CROSS-REFERENCE TO RELATED APPLICATIONS

This application claims priority to U.S. Provisional Patent Application No. 63/357,887, filed Jul. 1, 2022, which is incorporated by reference herein in its entirety for all purposes.

### FIELD OF THE INVENTION

The invention encompasses rapid diagnostic testing devices for testing of a biological sample for sepsis, and for differentiating between bacterial and viral pathogens. The device comprises a channeled construct, at least one lateral flow unit, and a cassette housing for the detection of biomarkers indicative of sepsis. The lateral flow unit is at least partially disposed in the cassette housing. The lateral flow unit comprises: a sample receiving zone, a conjugate zone and a detection zone. The sample receiving zone is operatively coupled to the channeled construct for receiving the biological sample comprising at least one analyte (e.g., a biomarker). The conjugate zone comprising a conjugate particle to bind the analyte is disposed adjacent to the first side of the sample receiving zone. The detection zone is disposed adjacent to the second side of the sample receiving zone and comprises at least one binding agent for detecting the analyte and includes quantum dots to amplify the signal for high sensitivity.

### BACKGROUND OF THE INVENTION

Sepsis is defined as a life-threatening organ dysfunction caused by a dysregulated host response. As a leading cause of worldwide mortality in children and adults, early recognition of sepsis is critical to reduce deaths, optimize health outcomes, and reduce the economic burden, but there are no objective early diagnostics. Children differ from adults in physiology, predisposing diseases, and sites of infection which necessitates differing diagnostic criteria and management strategies, as well as diagnostic biomarker panels. Furthermore, defining sepsis in the pediatric patient is made more difficult due to age specific vital signs and their tremendous physiologic reserve which often masks the seriousness of their condition. Accordingly, an early pediatric sepsis diagnostic would be quick, inexpensive, and provide sensitivity that gives the user confidence in the result irrespective of patient symptoms.

Common sepsis biomarkers have proved insufficient for accurately diagnosing sepsis, particularly across ages. The identification of accurate biomarker combinations to diagnose sepsis was the first step to develop point-of-care assays. Next steps include optimization of the proprietary lateral flow assay device, which includes components to accept the patient sample (including, but not limited to, whole blood, arterial or venous blood, capillary blood, plasma, serum, saliva, urine, tears, bronchoalveolar lavage, cerebrospinal fluid and/or tissue extracts), to purify the plasma component, to adhere selective capture antibodies and to linearly amplify the biomarker signals for clinical assessment.

The inventors have developed several point-of-care, multi-analyte lateral flow immunoassay utilizing novel pediatric and adult sepsis biomarkers together with highly efficient quantum dots to amplify the signal for high sensitivity.

### SUMMARY OF THE INVENTION

The inventors have developed multi-analyte lateral flow assays that consist of four to eight test strips in one test cartridge that allows for the sensitive detection of increases in protein levels of four to eight novel biomarkers indicative of either pediatric or adult sepsis.

In certain embodiments, the biomarkers include interleukin-6 (IL-6), interleukin-10 (IL-10), Granulocyte Colony Stimulating Factor (G-CSF), and calcitonin [CALCA; or procalcitonin (PCT)] due to the fold change in sepsis patients as well as early onset of change in IL-6, IL-10, and G-CSF.

The diagnostic assay is designed for point-of-care use in settings such as primary care offices, urgent care facilities, and emergency rooms. Patient samples to be used in the device would be capillary whole blood (e.g., finger prick) applied either directly to the test strips or via an anticoagulant-coated pipette. Upon sample addition, whole blood would be filtered to allow plasma to interact with quantum dot bioconjugates prior to flowing across the analytical membrane to allow for specific detection of host antigens. Results are interpreted visually with the use of an excitation light source and are typically available within about 1 to about 15 minutes. The assay may also utilize whole blood, arterial or venous blood, plasma, serum, saliva, urine, tears, bronchoalveolar lavage, cerebrospinal fluid and/or tissue extracts.

The diagnostic assay allows for early diagnosis of sepsis patients so healthcare providers can then (1) direct the patient/family to the emergency room; (2) have further tests ordered (imaging, blood/urine cultures, swabs, etc); and (3) and consider hospital admission for close observation, procedures and/or antibiotics/antivirals. A point-of-care sepsis diagnostic has the potential to reduce missed sepsis cases and those with late presentations thereby reducing morbidity and mortality.

In one embodiment, a rapid diagnostic testing device for rapid diagnostic testing of a biological sample is provided. The device comprises a channeled construct configured to receive at least a portion of the biological sample for rapid separation of one or more undesired components from the biological sample and forms at least partially purified biological sample; at least one lateral flow unit is operatively coupled to the channeled construct, and a cassette housing comprising a sample well, a plurality of rib structure, a first surface and a second surface, wherein the lateral flow unit is at least partially disposed in the cassette housing. The lateral flow unit comprises: a sample receiving zone operatively coupled to the channeled construct for receiving the partially purified biological sample from the channeled construct, wherein the partially purified biological sample comprises at least one analyte, and wherein the sample receiving zone comprises a first side and a second side; a conjugate zone adjacent to the first side of the sample receiving zone, wherein the conjugate zone comprises a conjugate particle for binding with the analyte; and a detection zone adjacent to the second side of the sample receiving zone, wherein the detection zone comprises at least one binding agent for detecting the analyte by capturing the analyte.

In one embodiment, the invention encompasses a diagnostic testing device for rapid diagnostic testing of a biological sample (e.g., whole blood or other biological samples) is provided.

In another embodiment, the device comprises a channeled construct configured to receive at least a portion of the biological sample; at least one lateral flow unit operatively coupled to the channeled construct; and a cassette housing comprising a sample well, a first surface and a second surface, wherein the lateral flow unit is at least partially disposed in the cassette housing. The lateral flow unit comprises: a sample receiving zone operatively coupled to the channeled construct for receiving at least a portion of the plasma from the channeled construct, wherein the plasma comprises at least one analyte or biomarker, and wherein the sample receiving zone comprises a first side and a second side; a conjugate zone disposed adjacent to the first side of the sample receiving zone, wherein the conjugate zone comprises a quantum dot-conjugate particle for binding with the analyte (biomarker) to form an analyte-conjugate complex; and a detection zone, wherein the detection zone comprises at least one binding agent for detecting the analyte by capturing the analyte and upon sample addition, whole blood is filtered to allow plasma to interact with quantum dot bioconjugates prior to flowing across the analytical membrane to allow for specific detection of host antigens.

In another embodiment, a diagnostic testing device for rapid diagnostic testing of a biological sample is provided herein. The device comprises a channeled construct configured to receive at least a portion of the blood sample for rapid separation of blood cells and plasma from the biological sample; at least one lateral flow unit operatively coupled to the channeled construct, and a cassette housing comprising a sample well; wherein the lateral flow unit is at least partially disposed in the cassette housing.

The lateral flow unit comprises: a sample receiving zone operatively coupled to the channeled construct for receiving at least a portion of the plasma from the channeled construct, wherein the portion of the plasma comprises at least one biomarker, a conjugate zone disposed adjacent to the sample receiving zone, wherein the conjugate zone comprises at least one quantum dot and an antibody for binding with the antigen to form an antigen-antibody complex; and a detection zone disposed adjacent to the second side of the sample receiving zone, wherein the detection zone comprises a test region comprising a secondary antibody disposed on the test region for detecting the antigen by capturing the antigen-antibody complex.

In certain embodiments, the invention encompasses a lateral flow immunoassay device comprising a lateral flow test strip, one or more quantum dots to which are conjugated antibodies for a specific biomarker, wherein the lateral flow strip has a sample pad and a wicking pad distal to the sample pad, a control line opposite the sample pad, wherein the device is capable of detecting the presence of an amount of the biomarker at picogram/mL and femtogram/mL concentrations in a biological sample, and wherein the biomarker is a biomarker for sepsis.

In certain embodiments, the biomarker for pediatric sepsis is IL-6, CALCA (or PCT), CSF3 (G-CSF), IL-1RA, IL-10, MCP-1, Elastase 2, or combinations thereof.

In certain embodiments, the biomarker for adult sepsis is IL-6, CALCA (or PCT), OSM, CH13L1, ST2, IL-10, IL-1RA, or combinations thereof.

In certain embodiments, the sepsis is pediatric sepsis.

In certain embodiments, the sepsis is adult sepsis

In certain embodiments, the lateral flow strip comprises a backing film, in which a piece of sample pad and absorbent pad are laminated at each end of the strip and a piece of membrane is laminated in the middle, and the quantum dots are immobilized in the membrane as the test line.

In certain embodiments, the device further comprises a cassette.

In certain embodiments, the cassette comprises a cover and a lateral flow strip holder.

In certain embodiments, the device comprises a single multiplex device comprising the following combination of biomarkers: IL-6, CALCA (or PCT), OSM, and CH13L1.

In certain embodiments, the device comprises a single multiplex device comprising the following combination of biomarkers: IL-6, CALCA (or PCT), OSM, and ST2.

In certain embodiments, the device encompasses a single multiplex device comprising the following combination of biomarkers: IL-6, CALCA (or PCT), IL-10, and IL1RA.

In certain embodiments, the device comprises a single multiplex device comprising the following combination of biomarkers: IL-6, CALCA (or PCT), CSF3 (G-CSM), and IL-1RA.

In certain embodiments, the device comprises a single multiplex device comprising the following combination of biomarkers: IL-6, IL-10, CSF3 (G-CSM), and IL-1RA.

In certain embodiments, the device comprises a single multiplex device comprising the following combination of biomarkers: IL-6, CSF3 (G-CSM), MCP-1, and Elastase 2.

In certain embodiments, the device comprises a single multiplex device comprising the following combination of biomarkers: PRTN3, NCF2, CXCL1, ST2 (IL-33), FDF21, and LBP. In certain embodiments, identification of the preceding biomarkers is indicative of a bacterial infection.

In certain embodiments, the device comprises a single multiplex device comprising the following combination of biomarkers: GRZB, MMP2, IL20, and CCL28. In certain embodiments, identification of the preceding biomarkers is indicative of a viral infection.

In another embodiment, the invention encompasses a method of detecting the presence of an amount of a sepsis biomarker using the lateral flow immunoassay device of claim 1, the method comprising contacting the lateral flow strip with a sample; flowing the sample over the one or more arrays; and detecting a quantum dot signal in the one or more arrays to thereby detect the presence of an amount of the sepsis biomarker.

### BRIEF DESCRIPTION OF THE DRAWINGS

FIG. 1 illustrates a sepsis biomarker time course following pathogen exposure.

FIG. 2 illustrates machine learning analyses of multiplex.

FIG. 3 illustrates ROC analyses of multiplex data.

FIG. 4A illustrates an example of an RDT device of the invention. The RDT device is configured to rapidly detect analytes present in a biological sample. The RDT device comprises a lateral flow unit test strip illustrated in FIG. 4B.

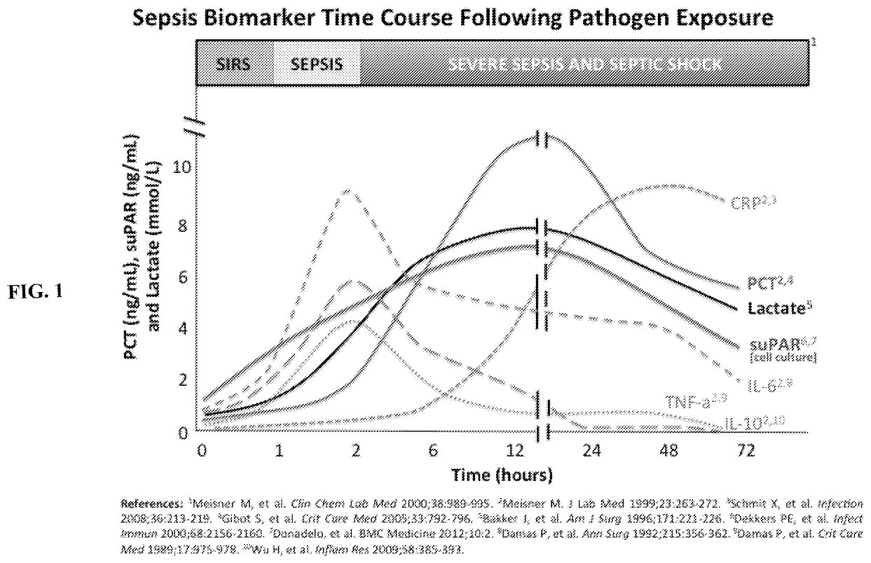

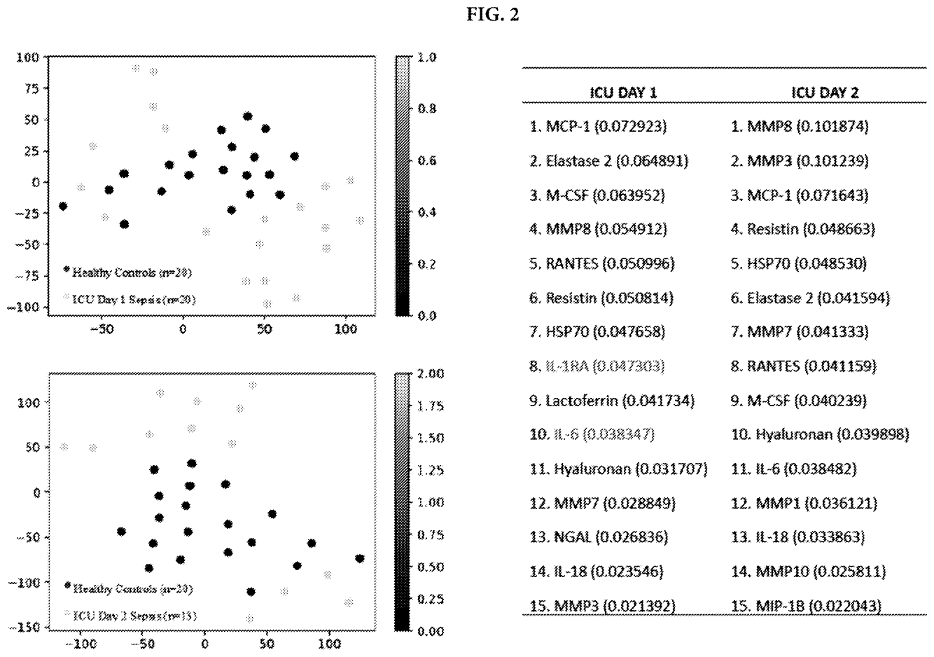

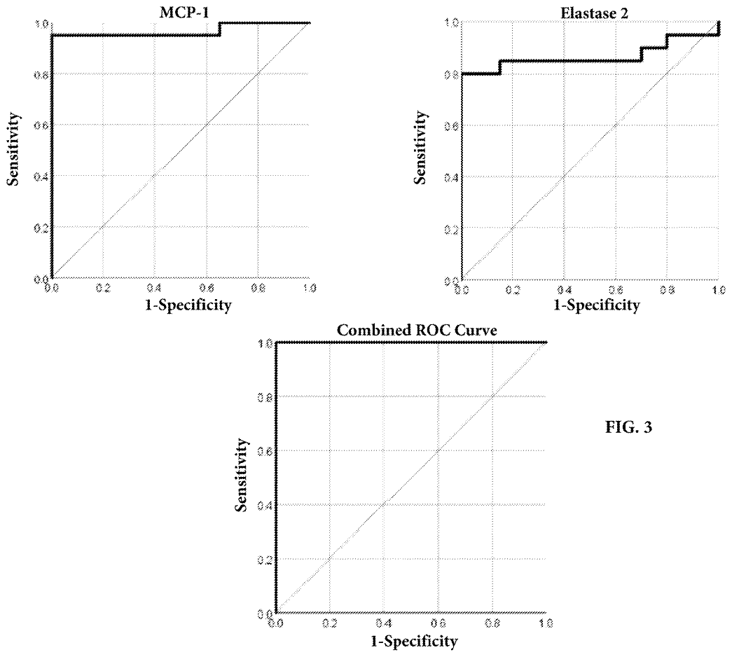

### DETAILED DESCRIPTION OF THE INVENTION

Rapid diagnostic tests (RDTs) or rapid diagnostic testing devices (RDT devices) broadly include lateral flow assays (LFAs) and/or flow through assays (FTAs). RDTs or RDT device using LFAs are provided herein, wherein the LFAs are used for detection of analytes, such as different biomarkers present in a biological sample by immuno-chromatographic antigen-detection tests. The immuno-chromatographic antigen-detection tests rely on capture of analytes (antigens) by quantum dot-labeled antibodies to produce a visible band on a lateral flow assay unit, such as a nitrocellulose test strip. The lateral flow assay unit is encased in a housing, referred to as a cassette. For RDTs, in one aspect, the quantum dot-labeled antibody or conjugate particle-coupled antibody binds to an analyte (antigen) such as a sepsis biomarker. The resultant analyte-antibody complex is further captured by the binding agents (secondary antibody) on a test line of the lateral flow unit, forming a visible test line in a result window of the RDT device that can be visually interpreted with the use of an excitation source.

In another aspect, the analytes bind to the antibodies on the test line forming analyte-antibody complex, which is further bound to conjugate particle-coupled antibody on the test line, forming a visible test line in the result window. In this case, a positive result is indicated by the presence of a test line. Presence of excess conjugate particles is desired, so that during detection, some of the conjugate particles are captured at the test line and continue to flow towards the second line of immobilized antibodies to a control line. This control line typically comprises a species-specific anti-immunoglobulin antibody, specific for the conjugate particle-coupled antibody. The control line gives information on integrity of the conjugate particle-coupled antibody and fluidics of the lateral flow unit.

To more clearly and concisely describe the subject matter of the disclosed application, the following definitions are provided for specific terms, which are used in the following description and the appended embodiments. Throughout the specification, exemplification of specific terms should be considered as non-limiting examples.

The singular forms “a”, “an” and “the” include plural referents unless the context clearly

dictates otherwise.

Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. In some instances, the approximating language may correspond to the precision of an instrument for measuring the value. Where necessary, ranges have been supplied, and those ranges are inclusive of all sub-ranges there between.

As used herein, the term “rapid diagnostic test” (RDT) refers to testing of a biological sample, which can be carried out at the point of care to obtain a fast diagnosis. A RDT is a medical diagnostic test that is quick and easy to perform and can be carried out even in the absence of laboratory techniques such as microscopy, enzyme-linked immunosorbent assay (ELISA) or polymerase chain reaction (PCR). RDTs provide results within less than an hour, typically in approximately 15 minutes. By way of a non-limiting example, RDTs for sepsis typically require about 15 minutes from the time of sample collection to the time of obtaining a result. It will be understood that the time required for an RDT depends on variables such as the type of sample, the amount of sample, the nature of the analyte and the like.

As used herein, the term “channeled construct” refers to a structure having pores, a first surface and a second surface. The first surface of the channeled construct is present at the top and second surface of the channeled construct present at the bottom of the channeled construct, when the channeled construct is vertically disposed on a lateral flow unit. A cross-section of the channeled construct may be larger at the first surface and relatively smaller at the second surface. The channeled construct may expand in more than one directions. By way of example, two or more separation elements may join to form a single unit of channeled construct. In a non-limiting example, the channeled construct may be in the shape of the letter “L.”

Embodiments of a rapid diagnostic testing (RDT) device for testing of a biological sample is provided herein. The RDT device is compatible with equipment-free, point of care (POC) analyte-separation and detection process. For example, the RDT device provides rapid diagnostic testing by immunochromatographic separation and detection.

According to embodiments of the present invention, the RDT device comprises a channeled construct, at least one lateral flow unit, and a cassette housing. The channeled construct is configured to receive at least a portion of a biological sample for rapid separation of undesired materials from the biological sample and forms at least partially purified biological sample. The lateral flow unit comprises a sample receiving zone, a conjugate zone, and a detection zone. The sample receiving zone comprises a first side and a second side.

Further, the sample receiving zone is operatively coupled to the channeled construct for receiving the partially purified biological sample from the channeled construct. The partially purified biological sample comprises at least one analyte or biomarker for sepsis, preferably a marker for sepsis in pediatric subjects. The conjugate zone is disposed adjacent to the first side of the sample receiving zone, wherein the conjugate zone comprises a conjugate particle for binding with the at least one analyte. The detection zone is disposed adjacent to the second side of the sample receiving zone. The detection zone comprises at least one binding agent including a quantum dot for detecting the at least one analyte by capturing the analyte. The cassette housing comprises a sample well, a plurality of rib structures, a first surface and a second surface. The lateral flow unit is operatively coupled to the channeled construct. Further, the lateral flow unit is at least partially disposed in the cassette housing.

As noted, the lateral flow unit and the channeled construct are operatively coupled to each other, wherein the term “operatively coupled” refers that the channeled construct and the lateral flow unit are coupled or connected when the RDT device is in operation. The channeled construct and the lateral flow unit are coupled or connected at least by a fluidic communication. The fluidic communication may include at least a fluid flow from the channeled construct to the lateral flow unit during operation of the device. For example, a plasma derived from the blood sample flows from the channeled construct to the lateral flow unit, which provides a fluidic communication under the operating conditions of the diagnostic testing device. In some embodiments, the channeled construct and the lateral flow unit may be in a physical contact.

The biological sample is a sample of blood, feces, sweat, saliva, mucous, milk, urine, semen, serum, plasma, capillary, sputum, tears, vaginal fluid, cerebrospinal fluid, bronchoalveolar lavage or tissue extract. In some embodiments, the RDT device is employed for testing of a blood sample. The RDT device is configured to separate blood cells from a blood sample to derive a plasma. Further, the plasma derived from the blood sample is used for detection of analytes using the quantum dot incorporated RDT device.

The channeled construct of the RDT device is disposed vertically relative to the at least one lateral flow unit. The term “vertically disposed relative to the lateral flow unit” means that the channeled construct is placed in a plane that is different from the plane comprising the lateral flow unit, and one of these planes is vertically disposed on the other. In such embodiments, the channeled construct is disposed at a right-angle on the sample window of the cassette housing of the RDT device. The plasma of the blood sample is transferred to the sample window from the channeled construct, where the channeled construct provides an initial separation of the blood sample by a physical separation, for example, size exclusion.

The lateral flow unit as employed for the present diagnostic testing device is a unit where liquid flows across the length of the lateral flow unit or lateral flow strip. The terms “lateral flow unit,” or “lateral flow assay strip” may be used interchangeably throughout the specification. Traditionally designed lateral flow units are composed of a variety of materials, each serving one or more purposes, overlapping onto one another, mounted on a backing substrate (e.g. backing card) using a pressure-sensitive adhesive.

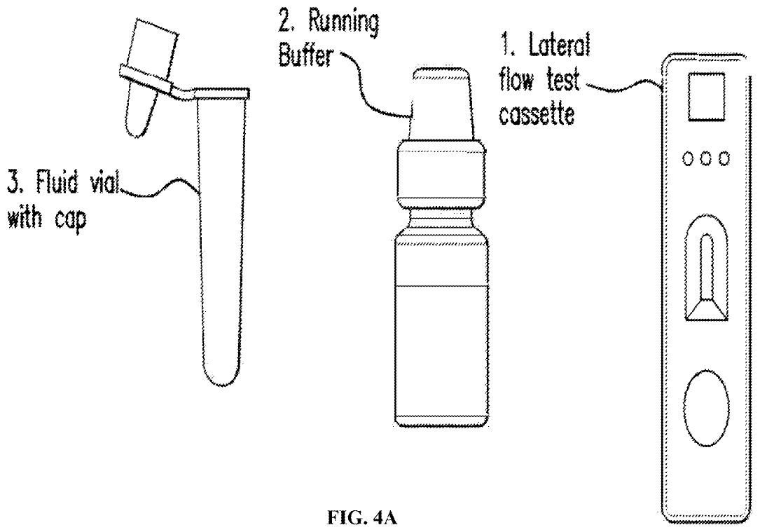

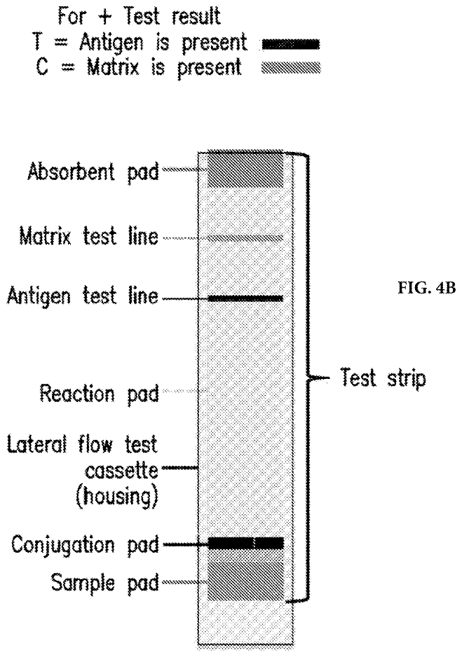

FIG. 4A illustrates an example of an RDT device of the present specification. The RDT device is configured to rapidly detect analytes present in a biological sample. The RDT device comprises a lateral flow unit test strip illustrated in FIG. 4B and a channeled construct. The lateral flow unit is configured to receive plasma derived from the blood sample. The lateral flow unit comprises a buffer reservoir, a conjugate zone, a sample receiving zone, and a detection zone. In some embodiments, the one or more zones of the lateral flow unit may be made of different materials. In some other embodiments, the various zones of the lateral flow unit is made of a single material.

In certain embodiments, the sample receiving zone of the lateral flow unit is operatively coupled to the channeled construct for receiving one or more components of the biological sample. The one or more components received by the channeled construct may include an analyte of interest, preferably a biomarker for sepsis, either pediatric or adult, which may be subsequently detected by the lateral flow unit. In embodiments where the biological sample is a blood sample, the sample receiving zone is configured to receive at least a portion of a plasma of the blood sample from the channeled construct, where the blood sample comprises at least one analyte.

In certain embodiments, one or more components of a biological sample, such as a plasma is received by the sample receiving zone, which may further be referred to as a “sample application pad” or a “sample pad.” The sample receiving zone has a first side and a second side. In one or more embodiments, the channeled construct is vertically disposed on or adjacent to the sample receiving zone such that the plasma from the channeled construct is received by the sample receiving zone of the lateral flow unit 10. The sample receiving zone may be present on a fiber glass, quartz, or a cellulose substrate.

The conjugate zone of the lateral flow unit is disposed adjacent to the first side of the sample receiving zone, leaving enough distance between the conjugate zone and the detection zone of the lateral flow unit. The conjugate zone comprises one or more conjugate particles for binding with at least one analyte present in the biological sample.

In certain embodiments, the conjugate zone is disposed adjacent to the first side of the sample receiving zone. The conjugate zone, may also be referred to as a “conjugate pad.” In one embodiment, the conjugate zone of the lateral flow unit comprises a conjugate particle. In another embodiment, the conjugate zone comprises a plurality of conjugate particles. In one or more embodiments, the number of conjugate particles present in the conjugate zone may be greater than the number of analytes present in the sample. In the conjugate zone, a conjugate particle may be immobilized on the conjugate zone 22 of the lateral flow unit 10. The conjugate particle binds to the analyte of the plasma derived from the blood sample received by the lateral flow unit or binds to binding agents disposed on the detection zone.

In various embodiments, the conjugate particle may include quantum dots. Implementations of the diagnostic system can use quantum dots as bio-labels. By replacing the signal marker on the LFIAs from ubiquitously used colloidal gold with quantum dots, the sensitivity and specificity can be improved significantly. Quantum dots also help achieve high performance. While colloidal gold nanoparticles are relatively large (>25 nm in size) biolabels used in sensing, the diagnostic system described in this specification can use quantum dots that are much smaller (<5 nm). These quantum dots can therefore, on an equivalent volume basis, offer more surface area. When conjugated with sensory proteins (i.e., antibodies), these biolabels can offer 3 orders of magnitude more binding sites for the analyte or biomarker, which leads to highly improved sensitivity. The novel biolabels also offer various advantages such as higher luminosity, a gamut of distinct colors for multiplexing tests, consistency in manufacturing, longer shelf life, and orders of magnitude cost savings over colloidal gold.

LFIAs labeled with quantum dots have high quantum yields (e.g., >35%), which enhances readability. In particular, test lines are easier to read and maintain readability much longer than colloidal gold-based LFIAs, which have low luminosity resulting from quantum yields <1%, making test lines difficult to read, which causes them to grow more faint with time. Quantum dot-labelled LFIAs also have a wide color gamut (potentially approaching or exceeding a million colors), which makes multiple analyte testing (multiplexing) possible. The limited color gamut (i.e., red and purple) of colloidal golf-based LFIAs makes multiplexing difficult. Furthermore, fluorescent nanoparticle-labelled LFIAs have a higher inherent stability over gold, which enhances durability, manufacturing consistency and shelf life of the LFIAs, and a lower cost of manufacture as compared to CG LFIAs.

The labelled LFIAs can use quantum dots such as CdSe/CdS tetrapod quantum dots)/metal (such as Ag) nanocluster technology that requires special manufacturing equipment (e.g., a microflow reactor) that prevents production of counterfeit test strips (i.e., because the quantum dot composition cannot be duplicated). The data reader can include a spectrometer for accurately detecting spectral signatures of the quantum dots and can be tuned to be sensitive only to quantum dots that produce specific spectral responses expected from the authorized quantum dots or can provide the spectral information for software analysis (e.g., by an app). The software in the app can be tuned to analyze the spectral information received from the data reader to be able to distinguish counterfeit test strips from authentic test strips. Thus, the test strips avoid problems with existing diagnostic tests, in which similar looking alternatives can be used to produce counterfeit test strips, as most of the components are readily available worldwide with little or no differentiation.

The RDT device may further include alternative conjugate reporters such as cellulose nanobeads (CNB), magnetic beads, fluorescence tags, chemiluminescence molecules, or various shapes of gold nanoparticles including nanospheres, nanorods, nanoshells. All such alternative conjugate reporters are contemplated within the scope of embodiments presented herein. The conjugate particle is conjugated to one of the components of the biological sample, a component of the lateral flow assay strip (such as binding agent), a biomolecule such as a protein. The protein may be an antigen or an antibody, depending on a format of the assay.

The detection zone is disposed adjacent to the second side of the sample receiving zone. The detection zone comprises at least one binding agent for detecting the at least one analyte by capturing the analyte. The detection zone may be constructed on a nitrocellulose membrane. In one embodiment, the detection zone may be formed by depositing one or more binding agents on the nitrocellulose membrane.

The analyte present in the plasma is detected in the detection zone of the lateral flow strip. In some embodiments, the detection zone comprises a test region. The test region is a sub-zone of the detection zone where binding agents are deposited. In some embodiments, the test region is a test line on the lateral flow strip. The binding agents are typically proteins, such as antibodies or antigens, which serve to capture the analyte or the analyte-conjugate complex as they migrate to the test region, depending on the assay requirement. The detection zone further comprises a control region. In some embodiments, the test region is a control line on the lateral flow strip. One or more binding agents having affinity towards the conjugate particles but no have affinity towards the analyte are deposited on the control region. In some embodiments, the binding agent is one or more of an antibody, or a labeled antibody. As used herein, “labeled antibody” includes any antibody coupled to an enzyme or a substrate, which is capable of changing color on exposure to a substrate, or reagent (such as an enzyme), respectively. As such, the antibody may be labeled with a quantum dot or a compound capable of producing chemiluminescence or fluorescence. In alternative embodiments, the antibody may be attached to a magnetic bead, a cellulose bead, a polymeric bead labeled with a dye, an affinity probe, and the like. In some embodiments, the binding agents are referred to as primary antibodies. In some alternative embodiments, the binding agents function as secondary antibodies.

In certain embodiments, the sample receiving zone and the conjugate zone of the lateral flow unit are present on a common substrate. In such embodiments, the conjugate particle in the conjugate zone may be present at one end of the common substrate and the sample receiving zone may be present at the opposite end of the common substrate. In one embodiment, the common substrate further comprises a detection zone. In certain other embodiments, at least one of the sample receiving zone, the conjugate zone, and the detection zone of the lateral flow unit is constructed on a substrate that is different than a substrate on which the other zones are constructed. In one or more embodiments, the common substrate is selected from a glass fiber, a nitrocellulose, or a quartz. In one embodiment, the common substrate is a nitrocellulose membrane.

The lateral flow unit further comprises a buffer reservoir disposed adjacent to the conjugate zone. The buffer reservoir is disposed such that when the RDT device is in operation, the buffer added to the buffer reservoir passes through the conjugate zone of the lateral flow unit. In some embodiments, the buffer reservoir is disposed on one end of the lateral flow unit. In one or more embodiments, a buffer solution may be added to the buffer reservoir. In some alternative embodiments, buffer reagents may be impregnated in the buffer reservoir, where the impregnated buffer reagents may be reconstituted as a buffer solution by adding water. The plasma sample comprising at least one analyte received by the sample receiving zone is chased with the buffer from the buffer reservoir to the different zones of the lateral flow unit. In operation, at least a portion of the buffer is passed from the conjugate zone to the sample receiving zone, and subsequently to the detection zone of the lateral flow unit. In one or more embodiments, the buffer reservoir comprises a non-lytic buffer.

In one or more embodiments, the lateral flow unit further comprises a wicking pad or an absorbent pad. The wicking pad is disposed adjacent to the detection zone. In some embodiments, the wicking pad is disposed adjacent to the detection zone and at the one end of the lateral flow unit. The wicking force of the wicking pad acts as a driving force to facilitate the buffer to flow through the lateral flow unit along a direction represented by reference numeral. The wicking pad draws the buffer to flow towards the wicking pad based on the strong wicking force. When excess conjugate particles move past the test region of the detection zone, the excess conjugate particles are entrapped in the wicking pad.

The RDT device may be operated in different ways, depending on the assay design, selection of conjugate particles, or selection of antibodies. In some embodiments, the conjugate zone comprises conjugate particles which are coupled to a primary antibody. In some of these embodiments, when buffer laterally flows from the buffer reservoir to the conjugate zone, the buffer remobilizes the dried primary antibody coupled-conjugate particles and subsequently flows to the sample receiving zone. Once the primary antibody-coupled conjugate particle and an analyte are in contact in the sample receiving zone, the primary antibody-coupled conjugate particle binds to the analyte to form a primary antibody-coupled conjugate-analyte complex.

The primary antibody-coupled conjugate-analyte complex along with remaining free conjugate particles and analyte particles may then migrate to the detection zone of the lateral flow unit. The detection zone is configured for detecting the analyte by capturing the primary antibody-coupled conjugate-analyte complex. In the detection zone, the binding agents, such as secondary antibodies disposed in the test region interacts with the conjugate-analyte complex. The secondary antibody binds to the primary antibody, where the primary antibody is coupled to the conjugate particle of conjugate-analyte complex. On binding of the secondary antibody to the primary antibody-coupled conjugate-analyte complex, a signal is generated at the test region, which is typically measured for detection of analyte.

In some alternative embodiments, the conjugate zone comprises conjugate particles which are coupled to secondary antibodies. In some of these embodiments, the test region of the detection zone comprises the binding agents that are primary antibodies specific to the analyte of interest. When buffer flows from the first end to the second end of the lateral flow unit, first, the analyte reaches the test region of the detection zone and is captured by the primary antibody disposed in the test region and forms primary antibody-analyte complex. Secondly, the secondary antibody coupled-conjugate particles traverse along with the buffer and reach the test region of the detection zone. The steps of capturing analyte by the primary antibody and traversing the secondary antibody coupled-conjugate particles along with the buffer from conjugate zone to the detection zone may occur simultaneously or consecutively. In the test region, once the secondary antibody coupled-conjugate particle is in contact with the primary antibody-analyte complex, the secondary antibody coupled-conjugate particle binds to the primary antibody-analyte complex. On binding of the secondary antibody coupled-conjugate particle to the primary antibody-analyte complex, a signal is generated at the test region, wherein presence of the signal is typically measured for detection of analyte.

The present RDT device of the subject specification advantageously allows for testing of larger volumes of samples compared to the sample used for currently available RDT devices. In some embodiments, a volume of blood sample employed for the present RDT device may be in a range from about 50 μL to about 200 μL. In some embodiments, a volume of the blood sample used for rapid diagnostic testing is in a range from about 75 μL to about 150 μL. In some other embodiments, a volume of the blood sample used for rapid diagnostic testing is in a range from about 90 μL to about 120 μL. The ability of an RDT device to process larger sample volume indicates that a larger volume of analyte reaches the lateral flow unit while using the present RDT device, which results in improving the signal intensity of the RDT device. In contrast, the sample read-out is affected by using the currently available RDT devices which are typically suitable for analyzing less amount of sample, such as about 5 μL of blood sample. By way of example, 100 μL of a blood sample is loaded to a channeled construct of an RDT device for rapid separation of blood cells and plasma from the blood sample as an initial step. The plasma from the 100 μL blood sample comprising at least one analyte is subsequently transferred to the lateral flow unit for analyte detection.

The present RDT device also avoids interference of red blood cells in sample-reading as the present RDT device is configured to exclude red blood cells from the blood sample prior to analyte detection. Thus, the detection of analyte is not affected by background noise from the presence of hemoglobin of red blood cells.

The RDT assay results using the RDT device are interpreted based on the presence or absence of a signal at the test region on the lateral flow unit. The RDT assay is determined visually or by using a reader to measure the signal intensity generated at the test region. The reader may include a plate reader, a spectrophotometer, a fluorescence spectrophotometer, reader for measuring chemiluminescence, and the like. The present quantum dot based RDT device of the subject specification generates higher signal intensity compared to the commercially available benchmark devices. The higher signal intensity is advantageous for detection of analytes because generally RDTs rely on visually detected changes in color of the test region on a lateral flow unit. A faint color change is not visually detectable and could lead to a false negative result on the RDT device.

In certain embodiments, provided herein is a channeled construct for separation of biological samples, such as blood. The construct is configured to receive and separates one or more components of a biological sample. In case of a blood sample, the channeled construct is configured to separate blood cells and plasma present in the blood sample. In some embodiments, the channeled construct comprises a size exclusion separation element. In some embodiments, the size exclusion separation element has a first surface distal from the lateral flow unit and a second surface proximal to the said lateral flow unit, when the channeled construct comprising the size exclusion separation element is vertically disposed on the lateral flow unit.

In some embodiments, the channeled construct comprises a size exclusion separation element. In one embodiment, the first surface of the size exclusion separation element is substantially planar with a raised edge surrounding the first surface of the size exclusion separation element. At least a portion of the second surface of the size exclusion separation element is in direct contact with a first surface of the lateral flow unit that comprises the sample receiving zone, conjugate zone, and detection zone. The channeled construct allows the plasma to flow through the channels and to reach the lateral flow unit of the RDT device.

In some embodiments, the size exclusion separation element may include a membrane, a chromatographic column, chromatographic beads, or a combination thereof, for rapid separation and delivery of a biological sample. In some other embodiments, the size exclusion separation element comprises progressively narrowing channels (elongated pores), which serve to physically filter out the red blood cells from a blood sample. In some embodiments, the size exclusion separation element is a porous membrane.

The size exclusion separation element, such as a porous membrane of the channeled construct ensures rapid separation of undesired materials from the biological sample to form at least partially purified biological sample comprising at least one analyte. Followed by purification, the channeled construct also delivers the partially purified biological sample to the lateral flow unit. In some embodiments, the porous membrane employed for the channeled construct is selected from an asymmetric porous membrane, a membrane comprising affinity surfaces, a membrane comprising hydrophobic cores, or a membrane comprising charged surfaces.

In some embodiments, the channeled construct comprises an asymmetric porous membrane having pores with asymmetric distribution. The asymmetric porous membrane has a first surface and a second surface, respectively. The asymmetric porous membrane is disposed on the channeled construct such that the upstream side and downstream side of the asymmetric porous membrane are aligned with the upstream side and downstream side of the channeled construct, respectively.

The pores having larger average pore-diameter as shown in first surface are on the upstream side of the asymmetric porous membrane act as a pre-filter for the separation of large particles, such as larger particles of blood sample. The pores having smaller average pore-diameter as shown in second surface present on the downstream side of the asymmetric porous membrane act as an exclusion zone or cut-off layer to further filter smaller particles from the fluid, such as the red blood cells to form a plasma. For example, the said distribution of pores allows a size-based filtration whereby larger particles/cells are retained in/on the membrane, while the smaller particles/analytes flow through the membrane. Asymmetric porous membranes may comprise a single layer or multiple layers. The pore size ratio of asymmetric porous membranes may vary depending on the sample being filtered.

In some embodiments, the asymmetric porous membrane is a polyethersulfone membrane, a polysulfone membrane, a glass fiber, a nylon membrane, a polyester membrane, a polycarbonate membrane, a polypropylene membrane, a polyvinylidene difluoride membrane, a cellulose membrane, a nitrocellulose membrane, a cellulose acetate membrane, a nitrocellulose mixed ester membrane, a polyurethane membrane, a polyphenylene oxide membrane, a poly(tetrafluoroethylene-co-hexafluoropropylene membrane, a cellulose phosphate membrane, a cellulose/silica gel paper, a borosilicate glass membrane, a quartz membrane, or a combination thereof. In a specific embodiment, the asymmetric porous membrane is an asymmetric polysulfone membrane. In another specific embodiment, the asymmetric porous membrane is an asymmetric polyethersulfone membrane.

In some embodiments, the size exclusion separation element of the channeled construct is designed as a plurality of conical shaped channels. Each of the channel has a smaller average pore diameters at the bottom of the channel 38 than the average pore diameter at the top of the channel 36. In some other embodiments, the channeled construct 34 comprises a size exclusion separation element, which is designed as a simple funnel filter for separating red blood cells. The red blood cells are unable to flow through the size exclusion separation element due to smaller pore diameter (at the bottom) than the diameter of the red blood cells. In some embodiments, each of the channels has an average pore diameter of about microns to about microns on the first surface and an average pore diameter of about 1 micron to about 3 microns on the second surface of the asymmetric porous membrane. In certain embodiments, the channeled construct is made of a polymer, a ceramic, a glass, a metal, or a combination thereof.

In some embodiments, a first surface of the asymmetric porous membrane 40 is coated with an anti-lysis coating. The first surface 36 of the asymmetric porous membrane 40 is defined as the surface where a biological sample is received in the channeled construct 34. The anti-lysis coating is used to stabilizes cells in a biological sample and prevents lysis and release of intracellular components of the cells. In some embodiments, the anti-lysis coating comprises a red blood cell stabilizer. In such embodiments, the blood separation membrane is coated with a red blood cell stabilizer that prevents lysis of the red blood cells. The prevention of cell lysis ensures minimal hemoglobin contamination during the detection step. In some embodiments, the channeled construct comprising a polymeric membrane coated with an anti-lysis coating. The polymeric membrane may be an asymmetric porous membrane.

In some alternative embodiments, the size exclusion separation element is disposed at a determined angle with respect to the lateral flow unit. In one example, the determined angle may be about 0. degree. In this example, the size exclusion separation element is disposed parallel to the lateral flow unit. In some of such embodiments, the parallelly disposed size exclusion separation element comprises a cellulose membrane, a nitrocellulose membrane, a glass fiber membrane, a quartz membrane, a borosilicate glass membrane, a mixed cellulose ester membrane, a polyvinylidene difluoride membrane, or a combination thereof, disposed laterally relative to the lateral flow unit. In such embodiments, the parallelly disposed size exclusion separation element allows for separation of a slower moving red blood cell front from a plasma front.

In some embodiments, the flow of the biological sample from the channeled construct to the lateral flow unit may be pressure-driven. The pressure may be generated after closure of the housing of the device, by capillary force, by gravity, in an electric field, or by any combination thereof. Similarly, the flow may be initiated by any such method that initiates contact of the biological sample with the sample pad, test region and/or control line of the lateral flow unit including manually applied pressure.

The lateral flow unit is at least partially disposed in a cassette housing, wherein the cassette housing ensures an efficient fluidic transfer from channeled construct to the lateral flow unit with a minimal loss of the biological sample. The use of the cassette housing is advantageous especially when a large volume of a blood sample containing numerous blood cells is applied to the RDT device. The lateral flow unit and the channeled construct are disposed in the cassette housing and arranged such that the chances of sample loss are reduced significantly.

In one or more embodiments, the sample well of the cassette housing comprises at least one wall forming a channel with a top aperture and a bottom aperture, where the bottom aperture includes a flange. The bottom aperture of the cassette housing is positioned to form a gap between the bottom aperture and the channeled construct. The flange of the bottom aperture is positioned to contact the channeled construct.

The cassette housing having a first surface and a second surface, further comprises a plurality of rib structure. In some embodiments, the plurality of rib structure includes two different series of rib structure. A series of rib structure extending from the first surface of the cassette housing, referred to herein as a “first series of rib structures”. The first series of rib structures are positioned adjacent to the sample well. The first series of rib structure is positioned such that a gap is formed between each of the rib structures and the sample well. The first series of rib structure is also in contact with the channeled construct.

Further, a series of rib structure extending from the second surface of the cassette housing is referred to herein as a “second series of rib structure.” The second series of rib structure extending from the second surface of the cassette housing are positioned on the second surface to form a gap between the rib structures and the channeled construct. The second series of rib structure is positioned such that a gap is formed between the rib structures and the lateral-flow unit.

In an alternative embodiment, the cassette housing is opened and a channeled construct is placed adjacent to the lateral flow unit such that one edge (proximal end) of the channeled construct contacts the lateral flow unit. The other edge of the channeled construct (distal end) placed adjacent to the sample well for receiving the biological sample. For example, the channeled construct employed for these embodiments may be L-shaped. The sample is received on the distal end of the L, wherein the heavier red blood cells are retained in the channeled construct while the plasma of the blood sample reaches the sample receiving zone of the lateral flow unit.

In certain embodiments, the cassette housing has a first surface and a second surface, wherein the first surface is an outer surface of the top section and the second surface is an outer surface of the bottom section of the cassette housing. The cassette housing includes a buffer well, disposed within the cassette housing. The buffer well is of a size, shape and position to permit receiving buffer to the RDT device during analyte detection process. The lateral flow unit is disposed in the cassette housing such that the buffer well is disposed adjacent to the buffer reservoir of the lateral flow unit. The buffer well is configured to flow buffer onto the buffer reservoir of the lateral flow unit. The cassette housing includes a test window. The test window includes an aperture positioned adjacent to the test region of the detection zone of the lateral flow unit. The buffer well is positioned adjacent to an end of the cassette housing and the test window is positioned centrally, adjacent to the sample well of the cassette housing.

The cassette housing also includes a sample well. The sample well is positioned between the buffer well and the test window. The sample well is of a size, shape and position to permit a biological sample to traverse through the sample well and to be deposited on to a channeled construct positioned within the cassette housing. In some embodiments, the sample well includes at least one wall forming a channel with a top aperture and a bottom edge/aperture. The sample well includes a bottom edge adjacent to a first surface of the channeled construct. The bottom edge of the sample well includes a flange that contacts the first surface of the channeled construct disposed in the cassette housing.

Depending on the embodiment, a cassette housing can be fabricated from materials selected for features such as weight, cost, durability, and chemical interactions with the interior features of the device. For example, in some embodiments the cassette housing is fabricated from a plastic material. For example, in some embodiments, the cassette housing is fabricated by a hydrophobic material. For example, in some embodiments the cassette housing is fabricated from a hydrophobic plastic material.

The rapid diagnostic testing devices described herein are applicable to a variety of RDTs including RDTs for detection of viruses, infectious diseases, bacteria, cancers, cardiac problems, animal diseases, sexually transmitted diseases, forensics, and the like.

The rapid diagnostic testing devices described herein may also be further adapted by including additional components such as colorimetric readers, photothermal readers, fluorescence readers, chemiluminescence readers, magnetic readers and the like. While typical RDTs are immune-chromatographic assays which rely on antibody conjugates, quantum dot labeled antibodies, or sandwich assays for detection, other methods of detection are contemplated within the scope of embodiments described herein including and not limited to colorimetric particles (metal particles, polymeric beads labeled with dyes, etc.), fluorescence, chemiluminescence, magnetic beads and the like. In addition to antibody capture, the analyte may be captured by techniques such as nucleotide/aptamer binding and such variants are contemplated as being within the scope of embodiments presented herein. It will be recognized that there are many types of assays such as competitive and non-competitive assays and such variations are also contemplated as being within the scope of embodiments presented herein. Further, multiple detection strips, and/or strips with multiple detection lines may be employed in the devices and methods described herein.

In some embodiments, the analyte is an antigen. In some embodiments, the analyte is a sepsis biomarker.

Table 1 illustrates multiplex significant sepsis biomarker data from Pediatric patients (<18 years of age).

TABLE 1

| | Healthy Controls | Sepsis Patients Day 1 | Sepsis Patients Day 2 | |
|---|---|---|---|---|
| Analyte | (n = 20) | (n = 20) | (n = 13) | P-value |
| CSF3 (G-CSF) | 0 (0, 56.97) | 498.26 (219.98, 5422.09) | 426.49 (104.57, 681.23) | <0.001 |
| IL-1RA | 8.32 (3.71, 17.67) | 231.23 (126.02, 1075.80) | 75.22 (29.60, 224.32) | <0.001 |
| IL6 | 0.67 (0.32, 1.68) | 288.22 (59.45, 3472.15) | 96.09 (24.35, 311.18) | <0.001 |
| IL8 | 1.70 (1.10, 3.58) | 12.79 (6.30, 249.72) | 8.70 (5.98, 180.31) | <0.001 |
| IL10 | 3.16 (0, 26.16) | 132.62 (30.15, 868.09) | 74.32 (23.20, 309.52) | <0.001 |
| IL18 | 39.95 (28.15, 58.54) | 87.32 (59.95, 116.55) | 86.58 (76.50, 115.00) | <0.001 |
| MCP-1 | 224.56 (187.10, 281.76) | 1273.33 (724.75, 3637.40) | 842.74 (617.09, 4619.61) | <0.001 |
| M-CSF | 14.25 (0.51, 42.50) | 240.44 (141.53, 474.90) | 209.70 (119.20, 539.35) | <0.001 |
| MIP-1β | 25.98 (19.61, 35.02) | 56.85 (38.73, 131.75) | 71.72 (38.33, 112.04) | <0.001 |
| TNFα | 22.20 (10.11, 53.54) | 119.82 (41.25, 384.94) | 73.57 (36.37, 339.42) | <0.001 |
| RANTES | 10590 (6150, 15275) | 4811 (1939, 6417) | 4121 (1356, 4597) | <0.001 |
| MMP3 | 7414 (3751, 10487) | 28604 (21388, 45900) | 28600 (19827, 40895) | <0.001 |
| MMP1 | 413.70 (367.57, 471.74) | 1374.53 (760.99, 3800.61) | 1099.36 (536.74, 1425.02) | <0.001 |
| MMP7 | 3892 (3279, 4360) | 7563 (4528, 11636) | 7771 (6136, 13026) | <0.001 |
| MMP10 | 444.2 (312.2, 616.1) | 2039.1 (971.6, 4170.0) | 2033.7 (1297.2, 4587.0) | <0.001 |
| Elastase2 | 3.01 (1.60, 4.19) | 21.23 (13.72, 58.22) | 19.79 (8.71, 33.05) | <0.001 |
| HSP70 | 21451 (19626, 32424) | 124090 (64321, 179781) | 144242 (69428, 239501) | <0.001 |
| MMP8 | 372.5 (217.7, 513.6) | 5089.1 (1129.6, 19915.9) | 6009.0 (1623.7, 15444.8) | <0.001 |
| Hyaluronan | 20.10 (14.85, 46.24) | 137.98 (85.20, 362.31) | 106.24 (55.31, 538.60) | <0.001 |
| NGAL | 60.86 (45.58, 95.74) | 223.05 (132.35, 1651.90) | 294.48 (134.41, 2063.79) | <0.001 |
| Resistin | 9.06 (6.41, 11.71) | 52.97 (17.42, 117.41) | 30.57 (17.70, 62.13) | <0.001 |
| MIG | 1295 (1013, 2772) | 3665 (1959, 13735) | 2776 (1947, 5235) | 0.004 |
| MMP9 | 16018 (13471, 18928) | 10235 (6335, 17943) | 8586 (5882, 15967) | 0.008 |
| MMP2 | 115962 (99215, 133619) | 98954 (82197, 106700) | 90882 (74505, 109475) | 0.009 |

Table 2 illustrates multiplex non-significant pediatric (<18 years of age) sepsis biomarker data.

TABLE 2

| | Healthy Controls | Sepsis Patients Day 1 | Sepsis Patients Day 2 | |
|---|---|---|---|---|
| Analyte | (n = 20) | (n = 20) | (n = 13) | P-value |
| IP10 | 108.31 (60.05, 163.97) | 373.02 (145.14, 4321.94) | 263.19 (81.05, 3557.59) | 0.024 |
| GM-CSF | 0 (0, 0) | 2.86 (0, 34.87) | 12.42 (0, 74.17) | 0.029 |
| Lactoferrin | 433.01 (289.20, 543.97) | 239.23 (118.83, 468.18) | 269.90 (168.92, 421.27) | 0.028 |
| IFNg | 4.04 (0.31, 7.91) | 14.75 (3.27, 31.71) | 8.08 (3.59, 20.80) | 0.047 |
| IL15 | 9.16 (3.63, 32.15) | 17.83 (10.88, 39.53) | 18.86 (10.07, 53.60) | 0.104 |
| IL12(p40) | 90.93 (49.55, 138.52) | 53.53 (33.25, 132.96) | 52.40 (28.35, 96.70) | 0.147 |
| Syndecan1 | 118.60 (58.24, 353.12) | 316.52 (138.28, 534.29) | 264.75 (125.72, 761.12) | 0.157 |
| Granzyme B | 2.99 (2.42, 8.16) | 10.21 (2.90, 14.60) | 3.60 (2.33, 15.09) | 0.168 |
| Thrombospondin-1 | 1008.78 (587.73, 1219.68) | 455.66 (209.23, 1026.87) | 540.89 (145.58, 1068.40) | 0.185 |
| VEGFA | 15.72 (4.97, 53.55) | 26.29 (16.54, 65.96) | 36.25 (15.65, 106.76) | 0.216 |
| IL7 | 0 (0, 0) | 0 (0, 0.48) | 0 (0, 0.60) | 0.224 |
| IL22 | 0 (0, 35.91) | 43.98 (0, 114.46) | 38.58 (0, 117.19) | 0.238 |
| Heparin sulfate | 25.16 (5.76, 61.49) | 7.15 (4.40, 33.85) | 5.96 (1.93, 59.42) | 0.239 |
| MMP12 | 517.60 (369.87, 599.81) | 976.94 (283.05, 2207.68) | 547.32 (272.16, 788.15) | 0.321 |
| IL17E/IL25 | 557.57 (185.72, 1174.19) | 685.27 (377.55, 2281.92) | 969.30 (303.59, 2563.56) | 0.358 |
| TNFβ | 0 (0, 8.84) | 2.78 (0, 17.87) | 0 (0, 14.87) | 0.414 |
| IL3 | 0 (0, 0.55) | 0 (0, 0) | 0 (0, 0.65) | 0.428 |
| POGF-AA | 87.94 (56.16, 180.66) | 148.53 (53.40, 303.39) | 152.65 (43.14, 251.89) | 0.442 |
| Eotaxin | 57.57 (44.73, 67.04) | 62.14 (49.26, 105.46) | 59.01 (43.56, 71.83) | 0.477 |
| PDGF-AB/BB | 1264.83 (445.21, 1875.60) | 652.24 (0, 1933.33) | 656.25 (0, 1628.67) | 0.489 |
| IL17F | 29.03 (11.10, 140.17) | 15.41 (4.34, 90.69) | 23.86 (2.42, 122.38) | 0.622 |
| IL2 | 0 (0, 0.94) | 0.04 (0, 1.87) | 0.20 (0, 1.97) | 0.625 |
| IL17A | 8.80 (1.52, 20.91) | 4.30 (2.24, 10.50) | 3.40 (1.13, 11.61) | 0.650 |
| IL 13 | 1.03 (0, 5.76) | 0.33 (0, 3.17) | 0 (0, 7.76) | 0.668 |
| Chondroitin sulfate | 9.29 (6.08, 15.00) | 7.86 (0.44, 20.99) | 10.32 (3.86, 27.88) | 0.701 |
| IL12(p70) | 2.88 (0, 9.54) | 2.74 (0.72, 6.84) | 1.91 (0, 7.78) | 0.746 |
| IFNa2 | 17.21 (2.11, 32.30) | 11.67 (5.14, 20.99) | 16.62 (4.92, 24.84) | 0.755 |
| MIP-1α | 20.92 (0, 75.25) | 34.31 (6.40, 73.36) | 28.28 (0, 87.41) | 0.763 |
| IL1α | 6.01 (1.77, 14.87) | 4.03 (1.54, 10.05) | 5.37 (2.10, 12.54) | 0.779 |
| IL4 | 0.08 (0, 4.06) | 0.45 (0, 2.68) | 0.41 (0, 6.46) | 0.865 |
| IL5 | 6.52 (3.01, 12.98) | 6.39 (2.66, 13.44) | 7.09 (2.51, 17.37) | 0.920 |
| IL1β | 13.94 (3.68, 24.38) | 10.45 (6.77, 20.70) | 9.26 (4.80, 24.13) | 0.949 |
| MMP13 | 692.19 (247.51, 1346.71) | 835.61 (228.91, 2336.98) | 812.89 (477.96, 1098.34) | 0.965 |
| EGF | 5.20 (0, 20.88) | 7.61 (0, 15.55) | 8.97 (0, 25.27) | 0.989 |

CONFIDENTIAL

Table 3 illustrates PEA significant pediatric (<18 years of age) sepsis biomarker data.

TABLE 3

| Rank | UniProt # | Protein | Sepsis Patients (n = 17) | Healthy Controls (n = 17) | AUC | P-value | Fold-Change |
|---|---|---|---|---|---|---|---|
| 1. | P [symbol] 5231 | IL6 | 72.93 (24.32, 743.31) | 0.34 (0.27, 0.56) | 1 | <0.001 | 214.50 |
| 2. | P [symbol] 1258 | CALCA | 60.00 (39.84, 75.57) | 0.80 (0.64, 0.99) | 1 | <0.001 | 75.00 |
| 3. | P [symbol] 0098 | CCL7 | 26.11 (9.28, 40.75) | 0.50 (0.48, 0.64) | 1 | <0.001 | 52.22 |
| 4. | P1 [symbol] 510 | IL1RA | 11.21 (5.21, 21.07) | 0.24 (0.23, 0.30) | 1 | <0.001 | 46.71 |
| 5. | P09919 | CSF3 (G-CSF) | 24.67 (7.08, 141.79) | 0.72 (0.58, 0.85) | 1 | <0.001 | 34.26 |
| 6. | Q01638 | IL1RL1 | 16.01 (5.87, 24.19) | 0.65 (0.51, 0.87) | 1 | <0.001 | 24.63 |
| 7. | P10145 | CXCL8 | 20.05 (6.46, 117.73) | 1.20 (1.03, 1.44) | 1 | <0.001 | 16.71 |
| 8. | O95407 | TNFRSF6B | 9.30 (4.56, 13.82) | 0.57 (0.45, 0.77) | 1 | <0.001 | 16.32 |
| 9. | P55773 | CCL23 | 3.37 (1.51, 5.41) | 0.33 (0.27, 0.35) | 1 | <0.001 | 10.21 |
| 10. | O43557 | TNFSF14 | 3.93 (2.76, 4.67) | 0.44 (0.36, 0.58) | 1 | <0.001 | 8.93 |
| 11. | Q99988 | GDF15 | 4.82 (2.55, 14.50) | 0.54 (0.48, 0.67) | 1 | <0.001 | 8.93 |
| 12. | P18428 | LBP | 13.36 (7.35, 22.90) | 1.75 (1.46, 2.43) | 1 | <0.001 | 7.63 |
| 13. | Q6PCB0 | VWA1 | 4.23 (3.19, 5.99) | 0.57 (0.43, 0.76) | 1 | <0.001 | 7.42 |
| 14. | Q9Y279 | VSIG4 | 3.32 (2.06, 8.51) | 0.45 (0.39, 0.59) | 1 | <0.001 | 7.38 |
| 15. | P [symbol] 1033 | TIMPI | 3.38 (2.49, 6.57) | 0.61 (0.52, 0.64) | 1 | <0.001 | 5.54 |
| 16. | Q9HAV7 | G [symbol] PELI | 4.94 (2.64, 8.81) | 0.92 (0.75, 1.01) | 1 | <0.001 | 5.37 |
| 17. | P10147 | CCL3 | 1.07 (0.68, 2.90) | 0.21 (0.18, 0.25) | 1 | <0.001 | 5.10 |
| 18. | O00220 | TNFRSF1DA | 2.25 (1.86, 3.38) | 0.47 (0.45, 0.55) | 1 | <0.001 | 4.79 |
| 19. | P14210 | HGF | 2.24 (1.76, 2.72) | 0.48 (0.44, 0.52) | 1 | <0.001 | 4.67 |
| 20. | O95633 | FSTL3 | 1.76 (1.20, 2.70) | 0.39 (0.35, 0.49) | 1 | <0.001 | 4.51 |
| 21. | P [symbol] MV8 | HSPA1A | 2.15 (1.11, 3.93) | 0.48 (0.38, 0.57) | 1 | <0.001 | 4.48 |
| 22. | Q02790 | FKBP4 | 4.69 (2.67, 6.24) | 1.06 (0.91, 1.20) | 1 | <0.001 | 4.42 |
| 23. | Q8NI22 | MCFD2 | 2.53 (1.77, 3.81) | 0.64 (0.58, 0.83) | 1 | <0.001 | 3.95 |
| 24. | P24394 | IL4R | 5.03 (3.73, 11.33) | 1.29 (1.13, 1.50) | 1 | <0.001 | 3.90 |
| 25. | P19438 | TNFRSF1A | 2.21 (1.60, 3.40) | 0.57 (0.54, 0.70) | 1 | <0.001 | 3.88 |
| 26. | P07858 | CTSB | 2.97 (1.99, 6.26) | 0.78 (0.62, 0.97) | 1 | <0.001 | 3.81 |
| 27. | O14763 | TNFRSF10B | 2.89 (1.88, 4.01) | 0.76 (0.71, 0.88) | 1 | <0.001 | 3.80 |
| 28. | P07306 | ASGR1 | 2.47 (2.18, 3.31) | 0.66 (0.53, 0.78) | 1 | <0.001 | 3.74 |
| 29. | P20333 | TNFRSF1B | 2.21 (1.76, 3.96) | 0.61 (0.50, 0.71) | 1 | <0.001 | 3.62 |
| 30. | P40933 | IL15 | 2.07 (1.28, 4.92) | 0.59 (0.50, 0.68) | 1 | <0.001 | 3.51 |
| 31. | Q6GTX8 | LAIR1 | 1.82 (1.13, 5.19) | 0.55 (0.50, 0.64) | 1 | <0.001 | 3.31 |
| 32. | P80188 | LCN2 | 1.81 (1.50, 3.69) | 0.64 (0.54, 0.75) | 1 | <0.001 | 2.83 |
| 33. | P [symbol] 7711 | CTSL | 1.41 (1.22, 2.11) | 0.50 (0.46, 0.59) | 1 | <0.001 | 2.82 |
| 34. | P [symbol] 0303 | NUCB2 | 1.55 (1.19, 2.12) | 0.59 (0.53, 0.72) | 1 | <0.001 | 2.63 |
| 35. | Q13232 | NME3 | 0.45 (0.39, 0.51) | 0.77 (0.72, 0.86) | 1 | <0.001 | 0.58 |
| 36. | Q8NFT8 | DNER | 0.32 (0.29, 0.34) | 0.62 (0.56, 0.71) | 1 | <0.001 | 0.52 |
| 37. | Q14956 | GPNMB | 0.54 (0.51, 0.66) | 1.12 (0.95, 1.31) | 1 | <0.001 | 0.48 |
| 38. | Q9NTU7 | CBLN4 | 0.57 (0.44, 0.72) | 1.27 (1.17, 1.46) | 1 | <0.001 | 0.45 |
| 39. | Q86SF2 | GALNT7 | 0.51 (0.43, 0.78) | 1.22 (1.11, 1.39) | 1 | <0.001 | 0.42 |
| 40. | P [symbol] 7949 | RET | 1.07 (0.93, 1.31) | 2.79 (2.58, 2.86) | 1 | <0.001 | 0.38 |
| 41. | Q12884 | FAP | 0.49 (0.33, 0.65) | 1.32 (1.07, 1.49) | 1 | <0.001 | 0.37 |
| 42. | Q9UP79 | ADAMTS8 | 0.34 (0.30, 0.47) | 1.08 (0.82, 1.39) | 1 | <0.001 | 0.31 |
| 43. | Q8IZP9 | ADGRG2 | 0.43 (0.32, 0.66) | 1.42 (1.15, 1.87) | 1 | <0.001 | 0.30 |
| 44. | O43508 | TNFSF12 | 0.38 (0.27, 0.46) | 1.28 (1.13, 1.45) | 1 | <0.001 | 0.30 |
| 45. | O60760 | HPGDS | 0.38 (0.30, 0.48) | 1.28 (0.89, 1.51) | 1 | <0.001 | 0.30 |
| 46. | Q9UGM5 | FETUB | 0.21 (0.14, 0.31) | 0.85 (0.71, 1.02) | 1 | <0.001 | 0.25 |
| 47. | O60911 | CTSV | 0.20 (0.14, 0.30) | 1.13 (0.95, 1.39) | 1 | <0.001 | 0.18 |
| 48. | O14788 | TNFSF11 | 0.20 (0.15, 0.29) | 2.66 (1.89, 3.90) | 1 | <0.001 | 0.08 |

[symbol]

indicates data missing or illegible when filed

Table 4 illustrates the top 25 Adult (>18 years of age) sepsis classifying proteins (PEA—non-converted values).

TABLE 4

| Protein | Healthy Controls | Sepsis Day I | Change | P Value |
|---|---|---|---|---|
| IL1RA | 3.94 (3.70-4.34) | 6.77 (6.33-7.17) | ↑ | <0.0001 |
| IL6 | 2.05 (1.63-2.27) | 7.40 (6.68-8.00) | ↑ | <0.0001 |
| FGF21 | 5.86 (4.61-6.18) | 8.96 (7.61-11.18) | ↑ | <0.0001 |
| OPG | 1.55 (1.22-1.75) | 2.90 (2.38-3.64) | ↑ | <0.0001 |
| GDF15 | 1.96 (1.65-2.23) | 3.98 (3.52-4.94) | ↑ | <0.0001 |
| PRTN3 | 2.59 (2.24-2.95) | 4.50 (4.04-5.04) | ↑ | <0.0001 |
| UPAR | 2.71 (2.54-2.98) | 3.94 (3.48-4.93) | ↑ | <0.0001 |
| CTSD | 1.66 (1.28-1.78) | 2.75 (2.17-3.21) | ↑ | <0.0001 |
| DAG1 | 1.41 (1.27-1.51) | 1.92 (1.82-2.20) | ↑ | <0.0001 |
| TPP1 | 4.26 (4.09-4.34) | 5.01 (4.85-5.73 | ↑ | <0.0001 |
| IL10 | 3.05 (2.77-3.47) | 5.79 (4.88-6.71) | ↑ | <0.0001 |
| CCL23 | 9.29 (8.66-9.53) | 11.17 (10.51-11.62) | ↑ | <0.0001 |
| CCL20 | 6.38 (5.81-6.74) | 9.15 (8.95-9.73) | ↑ | <0.0001 |
| SRP14 | 1.52 (1.27-1.86) | 3.31 (2.76-4.33) | ↑ | <0.0001 |
| CPA2 | 9.69 (9.16-9.82) | 7.34 (6.18-8.47) | ↓ | <0.0001 |
| GDF8 | 3.50 (3.25-4.10) | 1.50 (1.33-1.88) | ↓ | <0.0001 |
| NTRK3 | 6.74 (6.69-6.92) | 6.08 (5.75-6.34) | ↓ | <0.0001 |
| TGFα1 | 1.77 (1.49-1.94) | 2.71 (2.39-3.44) | ↑ | <0.0001 |
| S100A11 | 3.35 (3.19-3.55) | 4.34 (4.04-4.56) | ↑ | <0.0001 |
| WFDC2 | 8.54 (8.25-8.67) | 9.73 (9.46-10.59) | ↑ | <0.0001 |
| CXCL13 | 7.07 (6.79-7.40) | 8.67 (8.06-8.96) | ↑ | <0.0001 |
| ELOA | 0.41 (0.38-2.30) | 1.533 (1.26-2.10) | ↑ | <0.0001 |
| DSG4 | 2.52 (2.45-3.12) | 1.48 (0.67-2.08) | ↓ | <0.0001 |
| NBN | 0.86 (0.61-1.40) | 2.28 (1.98-2.82) | ↑ | <0.0001 |
| FGR | 1.51 (1.11-1.74) | 3.41 (3.04-4.18) | ↑ | <0.0001 |

Based on both multiplex and PEA data, the following biomarker combinations illustrated in Table 5 should be considered for a pediatric (<18 years of age) single multiplex sepsis biomarker diagnostic test.

TABLE 5

| | Protein | | Protein | | Protein |
|---|---|---|---|---|---|
| 1. | IL-6 | 1. | IL-6 | 1. | IL-6 |
| 2. | CALCA | 2. | IL-10 | 2. | CSF3 (G-CSF) |
| 3. | CSF3 (G-CSF) | 3. | CSF3 (G-CSF) | 3. | MCP-1 |
| 4. | IL-1RA | 4. | IL-1RA | 4. | Elastase 2 |

| Consistent among methods | Consistent among methods | Consistent among methods |
|---|---|---|
| Greatest fold-change | Greatest fold-change | Early/Rapid rise (IL-6) |
| Early/Rapid rise (IL-6, CALCA) | Early/Rapid rise (IL-6, IL-10) | Synergy for diagnoses |
| PCT may be substituted for CALCA | | (MCP-1 and Elastase 2) |

Legend
IL-6 = pro-inflammatory Cytokine, important mediator of fever and of the acute phase response.
IL-10 = anti-inflammatory cytokine with multiple, pleiotropic, effects in immunoregulation and inflammation
CSF3 (G-CSF) = a glycoprotein that stimulates the bone marrow to produce granulocytes
CALCA = a peptide hormone secreted by thyroid parafollicular cells (pro-calcitonin may be substituted)
IL-1RA = binds non-productively to the cell surface interleukin-1 receptor preventing inflammatory actions of IL-1
MCP-1 = recruits monocytes to areas of infection and inflammation
Elastase 2 = secreted by neutrophils during inflammation, and destroys bacteria and host tissue

Based on both multiplex and PEA data, the following biomarker combinations illustrated in Table 6 should be considered for an adult (≥18 years of age) single multiplex sepsis biomarker diagnostic test.

TABLE 6

| | Protein | | Protein | | Protein |
|---|---|---|---|---|---|
| 1. | IL-6 | 1. | IL-6 | 1. | IL-6 |
| 2. | CALCA | 2. | CALCA | 2. | CALCA |
| 3. | OSM | 3. | OSM | 3. | IL-10 |
| 4. | CH13L1 | 4. | ST2 | 4. | IL-1RA |

| Consistent among methods | Consistent among methods | Consistent among methods |
|---|---|---|
| Greatest fold-change | Greatest fold-change | Early/Rapid rise (IL-6, IL-10) |
| Early/Rapid rise (IL-6) | Early/Rapid rise (IL-6) | |

Legend
IL-6 = pro-inflammatory Cytokine, important mediator of fever and of the acute phase response.
CALCA = a peptide hormone secreted by thyroid parafollicular cells (pro-calcitonin may be substituted)
OSM = a pleiotropic cytokine that belongs to the interleukin 6 groups of cytokines
CH13L1 = a macrophage-secreted glycoprotein
ST2 = a member of the interleukin 1 receptor family and associated with cardiac stress
IL-10 = anti-inflammatory cytokine with multiple, pleiotropic, effects in immunoregulation and inflammation
IL-1RA = binds non-productively to the cell surface interleukin-1 receptor preventing inflammatory actions of IL-1

Table 7 lists potential biomarkers that distinguish bacterial from viral infections.

TABLE 7

| | |
|---|---|
| BACTERIAL > VIRAL | VIRAL > BACTERIAL |

| | | |
|---|---|---|
| PRTN3 | GRZB | CD40L |
| NCF2 | IL20 | CAS3 |
| CXCL1 | CCL28 | CAS8 |
| ST2 (IL-33) | MMP2 | IFNgama |
| FGF21 | PDCD1 | IP10 |
| LBP | KIR3DL | IL18 |

Based on Multiplex and PEA data, the Table 8 identifies 4 biomarker combination should be considered for a single multiplex.

TABLE 8

| | Protein | Rationale | | Protein | Rationale |
|---|---|---|---|---|---|
| 1. | PRTN3 | ↑ Bacterial/— viral | 1. | GRZB | ↑ viral/— bacterial |
| 2. | NCF2 | ↑ Bacterial/↓ viral | 2. | MMP2 | ↑ viral/↓ bacterial |
| 3. | CXCL1 | ↑ Bacterial/— viral | 3. | IL20 | ↑ viral/— bacterial |
| 4. | MMP2 | ↑ viral/↓ bacterial | 4. | CCL28 | ↑ viral/— bacterial |

| | |
|---|---|
| Moderately selective | Very selective |
| for bacterial illness | for viral illness |

While only certain features of the invention have been illustrated and described herein, many modifications and changes will occur to those skilled in the art. It is, therefore, to be understood that the appended embodiments are intended to cover all such modifications and changes as fall within the scope of the invention.

What is claimed is:

1. A lateral flow immunoassay device comprising a lateral flow test strip, one or more quantum dots to which are conjugated antibodies for a specific biomarker, wherein the lateral flow strip has a sample pad and a wicking pad distal to the sample pad, a control line opposite the sample pad, wherein the device is capable of detecting the presence of an amount of the biomarker at picogram/mL and femtogram/mL concentrations in a biological sample, and wherein the biomarker is a biomarker for sepsis.

2. The device of claim 1, wherein the biomarker for pediatric sepsis is IL-6, CALCA (or PCT), CSF3 (G-CSF), IL-1RA, IL-10, MCP-1, Elastase 2, or combinations thereof.

3. The device of claim 1, wherein the biomarker for adult sepsis is IL-6, CALCA (or PCT), OSM, CH13L1, ST2, IL-10, IL-1RA, or combinations thereof.

4. The device of claim 1, wherein the sepsis is pediatric sepsis.

5. The device of claim 1, wherein the sepsis is adult sepsis

6. The device of claim 1, wherein the lateral flow strip comprises a backing film, in which a piece of sample pad and absorbent pad are laminated at each end of the strip and a piece of membrane is laminated in the middle, and the quantum dots are immobilized in the membrane as the test line.

7. The device of claim 1, further comprising a cassette.

8. The device of claim 7, where the cassette comprises a cover and a lateral flow strip holder.

9. The device of claim 1, comprising a single multiplex device comprising the following combination of biomarkers: IL-6, CALCA (or PCT), OSM, and CH13L1.

10. The device of claim 1, comprising a single multiplex device comprising the following combination of biomarkers: IL-6, CALCA (or PCT), OSM, and ST2.

11. The device of claim 1, comprising a single multiplex device comprising the following combination of biomarkers: IL-6, CALCA (or PCT), IL-10, and IL1RA.

12. The device of claim 1, comprising a single multiplex device comprising the following combination of biomarkers: IL-6, CALCA (or PCT), CSF3 (G-CSM), and IL-1RA.

13. The device of claim 1, comprising a single multiplex device comprising the following combination of biomarkers: IL-6, IL-10, CSF3 (G-CSM), and IL-1RA.

14. The device of claim 1, comprising a single multiplex device comprising the following combination of biomarkers: IL-6, CSF3 (G-CSM), MCP-1, and Elastase 2.

15. The device of claim 1, comprising a single multiplex device comprising the following combination of biomarkers: PRTN3, NCF2, CXCL1, ST2 (IL-33), FDF21, and LBP.

16. The device of claim 15, wherein identification of the biomarkers is indicative of a bacterial infection.

17. The device of claim 1, comprising a single multiplex device comprising the following combination of biomarkers: GRZB, MMP2, IL20, and CCL28.

18. The device of claim 17, wherein identification of the biomarkers is indicative of a viral infection.

19. A method of detecting the presence of an amount of a sepsis biomarker using the lateral flow immunoassay device of claim 1, the method comprising contacting the lateral flow strip with a sample; flowing the sample over the one or more arrays; and detecting a quantum dot signal in the one or more arrays to thereby detect the presence of an amount of the sepsis biomarker.
